(19) United States
(12) Patent Application Publication
(10) Pub. No.: US 2012/0253278 A1
(43) Pub. Date: Oct. 4, 2012

(54) CATHETER AND PRODUCTION METHOD THEREOF

(76) Inventor: Hiroyoshi Ise, Shizuoka (JP)

(21) Appl. No.: 13/359,940

(22) Filed: Jan. 27, 2012

(30) Foreign Application Priority Data
Mar. 29, 2011 (JP) 2011-072799

(51) Int. Cl. A61M 25/10; B32B 38/18; A61B 17/22

(52) U.S. Cl. 604/96.01; 606/200; 606/194; 156/83

(57) ABSTRACT

A catheter is provided which allows medical treatment of the vascular aneurysm to be carried out with certainty and safely. The catheter includes a catheter main body including a first lumen for supplying and discharging expanding fluid therethrough and a second lumen into which an elongated instrument for the medical treatment is fitted, a balloon provided at a distal end portion of the catheter main body and communicating with the first lumen such that the balloon is expanded and contracted by supply and discharge of the expanding fluid thereto and therefrom, respectively, and a communication section open to an outer face of the balloon, communicating with the second lumen, and isolated from the inside of the balloon.

### CROSS-REFERENCE TO RELATED APPLICATION

This application claims priority under 35 U.S.C. §119 to Japanese Patent Application No. JP2011-072799 filed on Mar. 29, 2011, the entire content of which is hereby incorporated herein by reference in its entirety.

1. Field of the Invention

This invention relates to a catheter used to treat a vascular aneurysm and a production method of the catheter.

2. Background of the Invention

If a part of a blood vessel expands to form a vascular aneurysm, then there is the possibility that the vascular aneurysm may rupture to cause internal bleeding. Specifically, intracerebral hemorrhage caused by rupture of a vascular aneurysm in the brain is generally called stroke and sometimes leads to the death, and therefore, it is necessary to discover and treat intracerebral hemorrhage at an early stage.

Conventionally, as a treatment method of a vascular aneurysm, a method of filling a coil or agent for the embolus into a vascular aneurysm is known (refer to, for example, JP-T-2006-528030). Further, in recent years, a method of closing up an aneurysm entrance with a balloon catheter to fill a coil or agent with certainty is carried out.

In particular, according to this method, an instrument for the medical treatment such as a micro catheter for supplying a coil or agent is pushed into a vascular aneurysm, and then the aneurysm entrance is closed up with a balloon catheter and the instrument for the medical treatment is sandwiched into and fixed to the aneurysm entrance, whereafter a coil or agent is filled into the vascular aneurysm.

### SUMMARY OF THE INVENTION

However, with such a conventional method as described above, since the instrument for the medical treatment is sandwiched in the aneurysm entrance by a balloon catheter, there is the possibility that the coil for the embolus supplied in the aneurysm may protrude or agent for the embolus supplied in the aneurysm may leak out through a gap in which the instrument for the medical treatment is sandwiched. Further, if the sandwiching of the instrument for the medical treatment is insufficient, then there is the possibility that the instrument for the medical treatment may come off.

In order to solve such a subject as described above, it is one object of the present invention to provide a catheter which prevents protrusion of a coil or leakage of agent from a vascular aneurysm or coming off of an instrument for the medical treatment inserted in a vascular aneurysm so that the medical treatment of the vascular aneurysm can be carried out with certainty and safely.

In order to achieve the object described above, according to one form of the present invention, a catheter includes a catheter main body including a first lumen for supplying and discharging expanding fluid therethrough and a second lumen into which an elongated instrument for the medical treatment is fitted, a balloon provided at a distal end portion of the catheter main body and communicating with the first lumen such that the balloon is expanded and contracted by supply and discharge of the expanding fluid thereto and therefrom, respectively, and a communication section open to an outer face of the balloon and communicating with the second lumen in a state in which the communication section is isolated from the inside of the balloon.

In order to achieve the object described above, according to one form of the present invention, a production method of a catheter includes a preparation step of placing a tube for a balloon, which has a hollow shape and is expandable, into a molding die which has a cavity and an extruding portion which projects into the cavity, a molding step of injecting pressurized fluid into the tube for the balloon to expand the tube for the balloon to mold the balloon and a communication section open to an outer face of the balloon and isolated from the inside of the balloon, and an attachment step of attaching the balloon and the communication section to a distal end portion of a catheter main body which includes a first lumen for supplying and discharging expanding fluid therethrough and a second lumen into which an elongated instrument for the medical treatment is fitted thereby to establish communication of the inside of the balloon with the first lumen and communication of the communication section with the second lumen.

With the catheter of the form described above, since an instrument for the medical treatment can be inserted into a vascular aneurysm through the second lumen and the communication section while the balloon is kept in contact with the aneurysm entrance, protrusion of a coil or leakage of agent from the vascular aneurysm can be prevented. Further, since not the instrument for the medical treatment is inserted from between the inner wall of the aneurysm entrance and the outer surface of the balloon and sandwiched but the instrument for the medical treatment can be inserted into the vascular aneurysm through the second lumen and the communication section, coming off of the instrument for the medical treatment arising from insufficient sandwiching as in the prior art does not occur. Further, when the catheter and the instrument for the medical treatment are disposed in the proximity of the aneurysm entrance, since the instrument for the medical treatment is accommodated in the second lumen of the catheter, it is only necessary to take an outer diameter of the catheter with respect to the inner diameter of the blood vessel into consideration. Accordingly, the catheter can carry out a medical treatment for the vascular aneurysm reliably and safely.

Further, if the opening of the communication section formed on the outer face of the balloon is a small hole which restricts a change of the direction of the instrument for the medical treatment fitted in the opening, then since the direction of the instrument for the medical treatment inserted in the vascular aneurysm from the communication section is less likely to change, a coil or agent can be supplied stably into the vascular aneurysm.

Further, if the opening of the communication section formed on the outer face of the balloon is an elongated hole elongated along an axial direction of the catheter main body, then it is easy to direct the instrument for the medical treatment inserted in the vascular aneurysm from the communication section to a desired direction to supply a coil or agent to an aimed place in the vascular aneurysm. Therefore, the workability is good.

Further, if the opening of the communication section extends to an end portion of the balloon, then it is easy to move the instrument for the medical treatment inserted in the vascular aneurysm from the communication section over a wide range to supply a coil or agent to an aimed place in the vascular aneurysm. Therefore, the workability is good.

Further, if the expanding fluid is supplied further into the balloon in the expanded state to further expand the balloon so that the balloon blocks the opening of the communication section, then since the instrument for the medical treatment is retained in a desired direction by closing of the opening, a coil or agent can be supplied stably to an aimed place in the vascular aneurysm.

Further, if the communication section has a groove formed on an inner face thereof in such a manner as to extend in a direction intersecting with the axial direction of the catheter main body, then when the opening is closed, since the instrument for the medical treatment is fitted into the groove, the instrument for the medical treatment is less likely to be crushed but is retained stably.

Meanwhile, according to the production method of the catheter described above, a catheter which can carry out a medical treatment of a vascular aneurysm reliably and stably can be produced.

Further, a method for medically treating a vascular aneurysm includes a step of inserting a balloon catheter, which includes a catheter main body including a first lumen for supplying and discharging expanding fluid therethrough and a second lumen into which an elongated instrument for the medical treatment is fitted, a balloon provided at a distal end portion of the catheter main body and communicating with the first lumen such that the balloon is expanded and contracted by supply and discharge of the expanding fluid thereto and therefrom, respectively, and a communication section open to an outer face of the balloon and communicating with the second lumen in a state in which the communication section is isolated from the inside of the balloon, into a blood vessel until the balloon catheter reaches a place in the proximity of an opening of the vascular aneurysm, a step of expanding the balloon to block the opening of the vascular aneurysm, and a step of inserting the instrument for the medical treatment into a lumen of the vascular aneurysm from the communication section of the balloon catheter and filling the lumen of the vascular aneurysm using the instrument for the medical treatment, then since the instrument for the medical treatment can be inserted into the vascular aneurysm through the second lumen and the communication section while the balloon is kept in contact with the aneurysm entrance, protrusion of a coil or leakage of agent from the vascular aneurysm can be prevented. Further, since not the instrument for the medical treatment is inserted from between the inner wall of the aneurysm entrance and the outer surface of the balloon and sandwiched but the instrument for the medical treatment can be inserted into the vascular aneurysm through the second lumen and the communication section, coming off of the instrument for the medical treatment arising from insufficient sandwiching as in the prior art does not occur. Further, with the method described above, when the catheter and the instrument for the medical treatment are disposed in the proximity of the aneurysm entrance, since the instrument for the medical treatment is accommodated in the second lumen of the catheter, only it is necessary to take an outer diameter of the catheter with respect to the inner diameter of the blood vessel into consideration. Accordingly, a medical treatment for the vascular aneurysm can be carried out reliably and safely by the method described above.

### BRIEF DESCRIPTION OF THE DRAWINGS

FIG. 1 is a sectional view showing a catheter of a first embodiment.

FIG. 2 is an enlarged longitudinal sectional view showing a distal end portion of the catheter of the first embodiment.

FIG. 3 is an enlarged longitudinal sectional view showing a proximal end portion of the catheter of the first embodiment.

FIGS. 4A-4F are views showing a particular structure of the distal end portion of the catheter of the first embodiment.

FIGS. 5A-5D are views showing a structure of a molding die for molding a balloon of the first embodiment.

FIGS. 6A-6C are process drawings illustrating a molding method of the balloon of the first embodiment.

FIG. 7 is an explanatory view showing the catheter of the first embodiment in a used state.

FIGS. 8A-8F are views showing a particular structure of a distal end portion of a catheter of a second embodiment.

FIGS. 9A and 9B are views showing a structure of essential part of the catheter of the second embodiment.

FIGS. 10A-10F are views showing a particular structure of a distal end portion of a catheter of a third embodiment.

### DETAILED DESCRIPTION OF THE PREFERRED EMBODIMENTS

In the following, embodiments of the present invention are described with reference to the drawings. It is to be noted that the dimensional ratio of the drawings is sometimes exaggerated and different from an actual ratio for the convenience of illustration.

### First Embodiment

### (Catheter)

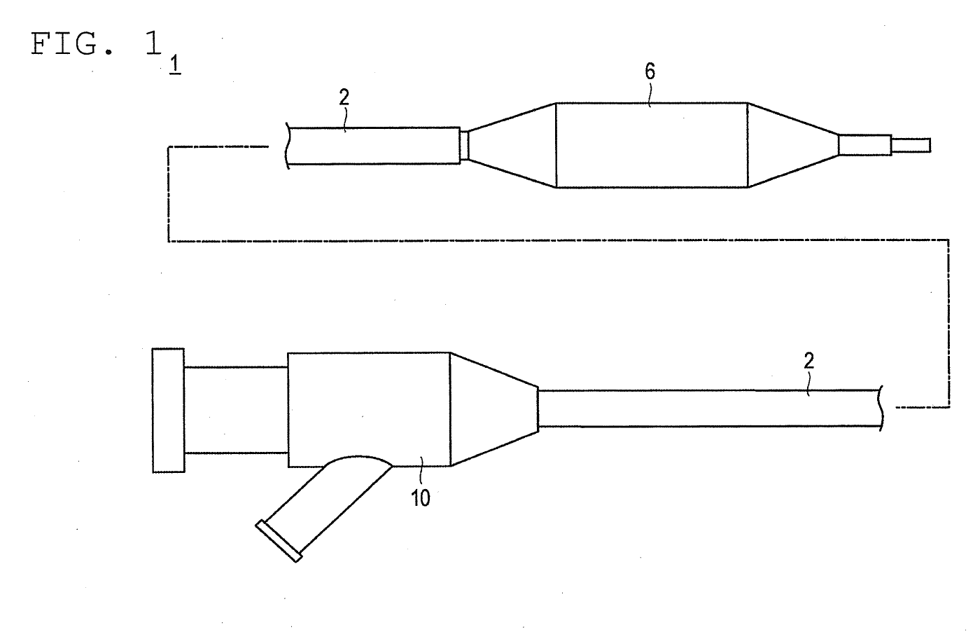

Generally describing with reference to FIG. 1, a catheter 1 according to the present embodiment is a catheter of the over-the-wire (over the wire: OTW) type wherein a guide wire extends from a hand to a distal end, and has a catheter main body 2 having a form of a tube and having flexibility, a hub 10 provided at a proximal end portion of the catheter main body 2, and a balloon 6 provided at a distal end portion of the catheter main body 2.

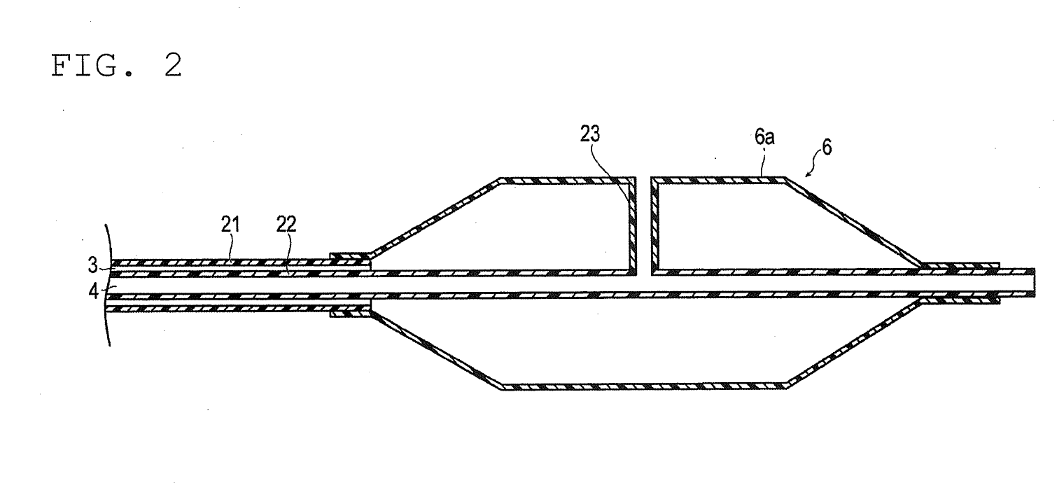

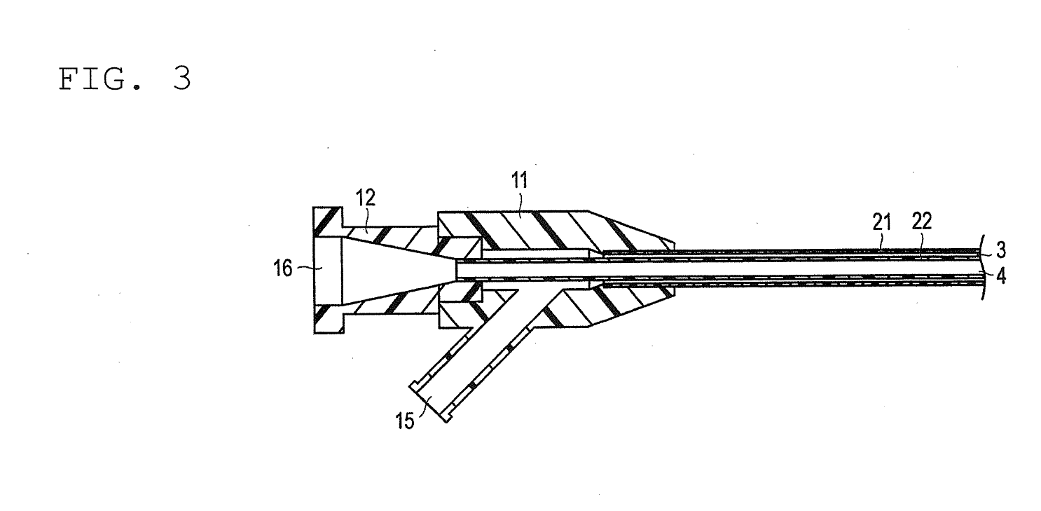

As shown in FIGS. 2 and 3, the catheter main body 2 has an outer tube 21 having a form of a tube which is open at a distal end and a proximal end thereof, and an inner tube 22 having a form of a tube which is open at a distal end and a proximal end thereof and provided inside the outer tube 21.

The outer tube 21 is formed coaxially on the outer side of the inner tube 22 and has a proximal end at a position retreating from the distal end portion of the inner tube 22. The inner diameter of the outer tube 21 is formed greater than the outer diameter of the inner tube 22, and a first lumen 3 for supplying and discharging expanding fluid to and from the balloon 6 therethrough is formed between the outer tube 21 and the inner tube 22.

As a material for configuring the outer tube 21, preferably a material having some flexibility is used, and as such material, for example, polyethylene, polypropylene, polybutene, ethylene-propylene copolymer, ethylene-vinyl acetate copolymer, ionomer, polyolefin such as mixture of two or more of the substances mentioned, plasticized polyvinyl chloride resins, polyamide, polyamide elastomer, polyester, polyester elastomer, polyurethane, thermoplastic resin such as fluorocarbon resin, silicone rubber, latex rubber and so forth can be used.

The inner tube 22 has a second lumen 4 into which an elongated instrument for the medical treatment such as a micro catheter or a coil transmission device can be fitted. The material from which the inner tube 22 is configured is a material similar to that of the outer tube 21 or any of various metal materials such as stainless steel, a stainless stretchable alloy, or a Ni—Ti alloy. A communication section 23 for introducing an instrument for the medical treatment fitted in the inner tube 22 to the inside of a vascular aneurysm is connected in a branched state.

The hub 10 has an outer tube hub 11 having a first opening 15, and an inner tube hub 12 having a second opening 16. The inner tube hub 12 is disposed on the proximal end side with respect to the outer tube hub 11 and fixed to the outer tube hub 11.

The outer tube hub 11 is fixed to a proximal end portion of the outer tube 21. As means for the fixation, adhesion by a bonding agent, heat seal, fixation by a fastener (not shown) and so forth are available. The first opening 15 is communicated with the first lumen 3 and functions as a port for introducing expanding fluid for expanding the balloon 6 into the first lumen 3 and discharging the expanding fluid from the first lumen 3.

The inner tube hub 12 is fixed to a proximal end portion of the inner tube 22. As means for the fixation, adhesion by a bonding agent, heat seal, fixation by a fastener (not shown) and so forth are available. The second opening 16 is communicated with the second lumen 4 and functions as a guide wire insertion port for fitting a guide wire into the second lumen 4. Further, the second opening 16 is communicated with the second lumen 4 and the communication section 23 and functions as a medical treatment instrument port for fitting an instrument for the medical treatment into the communication section 23 through the second lumen 4.

As a material from which the outer tube hub 11 and the inner tube hub 12 are configured, a thermoplastic resin such as polycarbonate, polyamide, polysulfone, polyarylate, or methacrylate-butylene-styrene copolymer is used suitably.

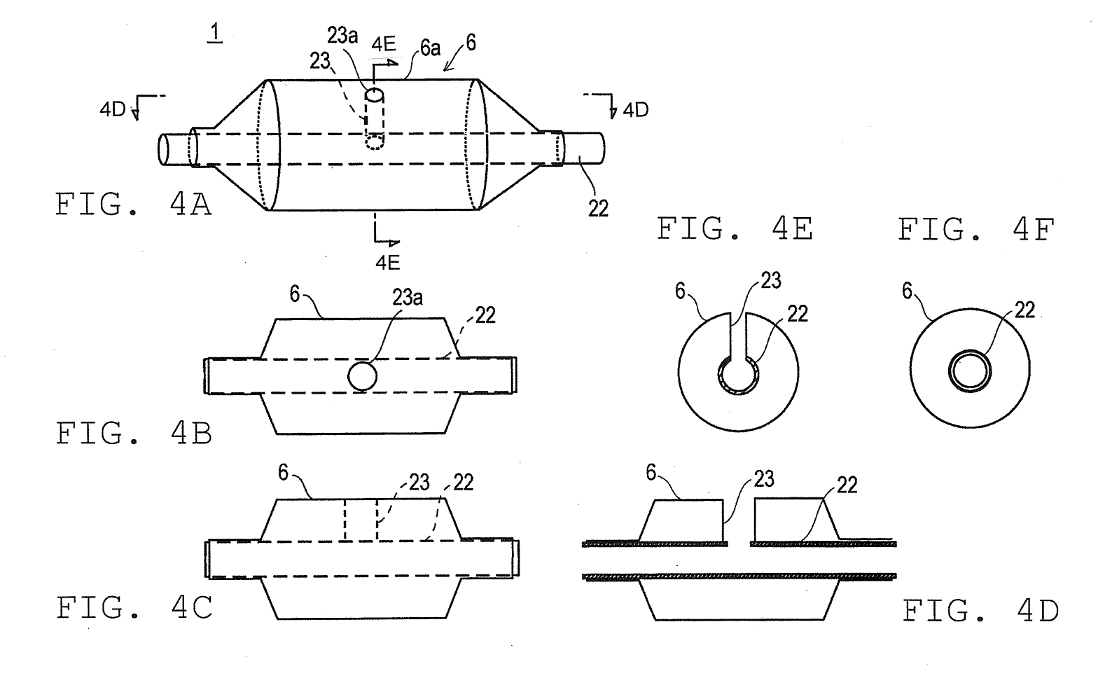

The communication section 23 is open to an outer face of the balloon 6 and is communicated with the second lumen 4 in a state in which it is spaced away from the balloon 6 without being communicated with the inside of the balloon 6. As shown in FIG. 4, an opening 23a of the communication section 23 on the outer face of the balloon 6 is such a small hole as to restrict variation of the direction of an instrument for the medical treatment fitted into the opening 23a.

The communication section 23 has a form of a tube, and the inner diameter of the communication section 23 is formed, for example, equal to or smaller than the inner diameter of the inner tube 22. In order to facilitate introduction of an instrument for the medical treatment, the cross section of the communication section 23 may have such a shape that the opening thereof increases from a connecting portion of the communication section 23 to the inner tube 22 to a balloon outer face (tapering like an inverted funnel shape). In the present embodiment, the inner tube 22 and the communication section 23 are formed separately from each other, and are connected to each other by a bonding agent or heat seal or the like.

The balloon 6 is formed for expansion and exhibits, in a state in which it is not expanded, such a state that it is contracted or folded on an outer periphery of the inner tube 22. The balloon 6 has a cylindrical portion 6a having a cylindrical shape. The balloon 6 closes up the entrance of a vascular aneurysm with the cylindrical portion 6a.

The balloon 6 is closed up and sealed at a distal end portion thereof by a bonding agent or heat seal or the like to an outer peripheral face of a distal end portion of the inner tube 22, and is similarly closed up and sealed at a proximal end portion thereof to an outer peripheral face of a distal end portion of the outer tube 21. The inside of the balloon 6 is communicated with the first lumen 3 such that expanding fluid (liquid) such as physiological salt solution and contrast agent can flow into the inside of the balloon 6 through the first lumen 3. The balloon 6 is expanded by the expanding fluid flowing in, and is placed into a contracted or folded state by discharging the expanding fluid flowed in. The first lumen 3 is not communicated with the communication section 23.

At a predetermined location of the inner tube 22 covered with the balloon 6, an X-ray impermeable marker (not shown) is provided. The X-ray impermeable marker is configured from a winding wire of an X-ray impermeable material, and is formed preferably, for example, from platinum, gold, tungsten, iridium or an alloy of them, and more preferably from platinum, gold, or platinum-iridium alloy. By forming the X-ray impermeable marker from any of those materials, a clear contrast image can be obtained under X-ray radioscopy, and therefore, the position of the cylindrical portion 6a of the balloon 6 can be confirmed readily under X-ray radioscopy.

### (Production Method of the Catheter)

Generally describing, the production method of the catheter 1 of the present embodiment includes a preparation step of placing an expandable parison (tube for a balloon) having a hollow shape into a molding die for molding the balloon 6 and the communication section 23, a forming step of inflating the parison to mold the balloon 6 and the communication section 23 after the preparation step, and an attachment step of attaching the balloon 6 and the communication section 23 to the catheter main body 2 after the molding step.

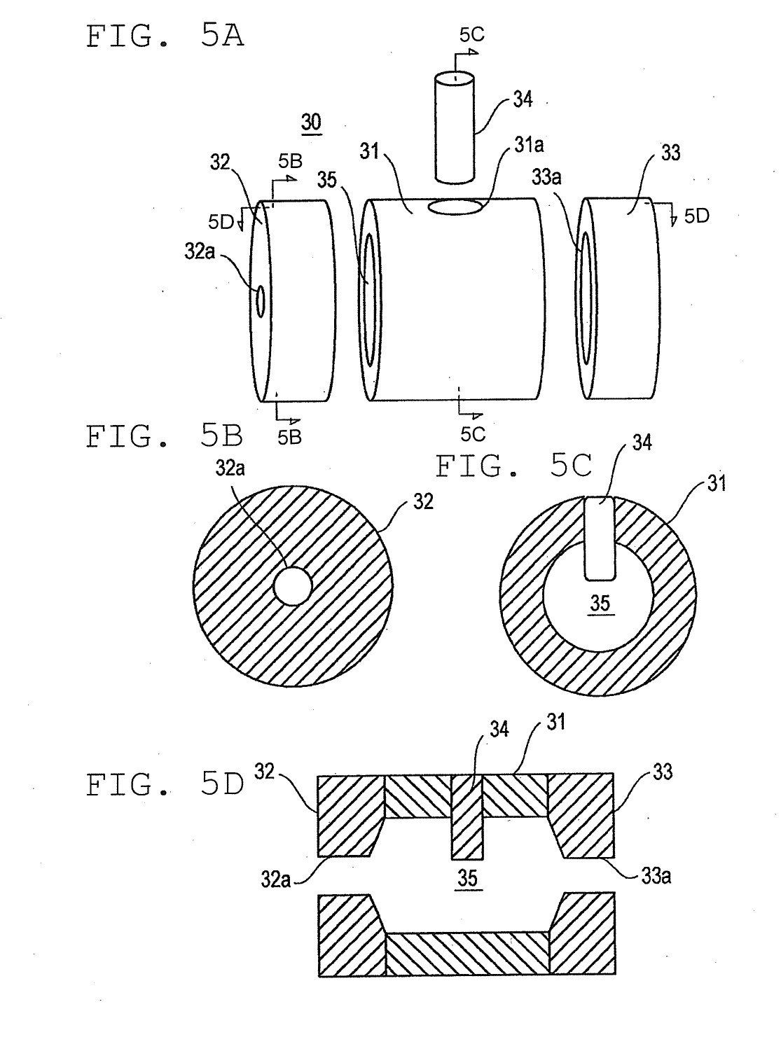

As shown in FIGS. 5A-5D, a molding die 30 for molding the balloon 6 includes a central die 31 of a cylindrical shape for molding the cylindrical portion 6a of the balloon 6, a left side die 32 and a right side die 33 disposed on the opposite sides of the central die 31 in an axial direction for cooperating with the central die 31 to form a cavity 35 for molding the balloon 6, and a core die 34 (extruding portion) incorporated so as to project into the cavity 35 to mold the communication section 23.

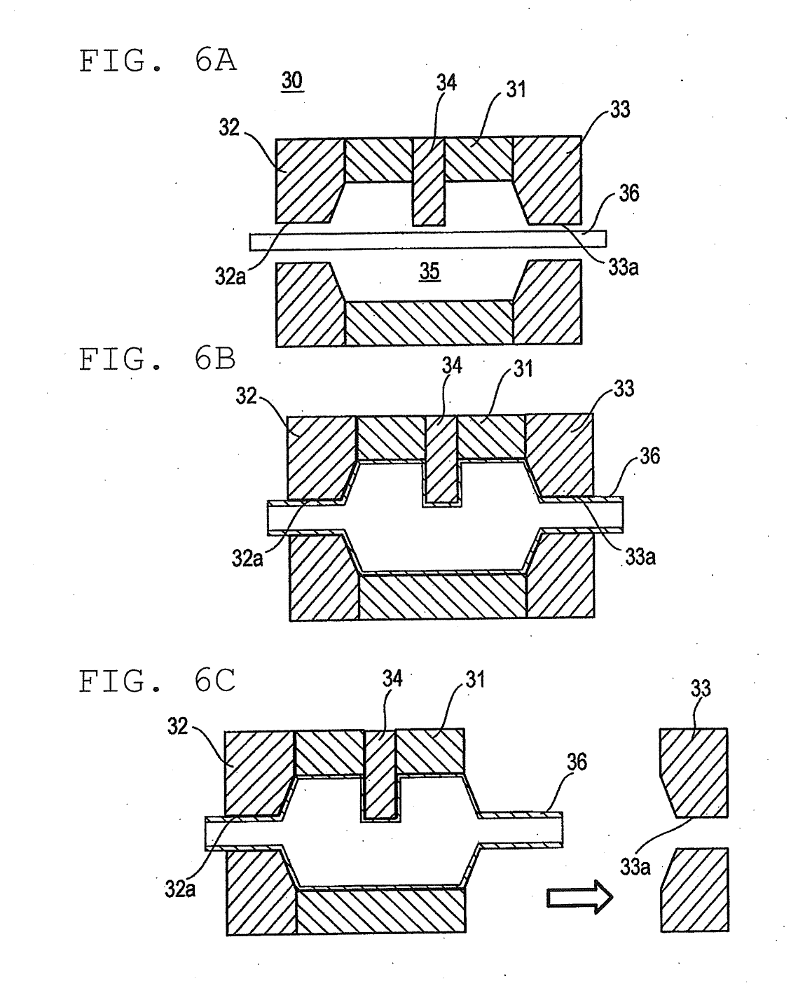

At an upper portion of a circumferential wall of the central die 31, a core hole 31a for extruding the core die 34 into the cavity 35 therethrough so as to be disposed in the cavity 35 is formed such that it extends in a diametrical direction therethrough. The left side die 32 and the right side die 33 have substantially disk shapes, and fitting holes 32a and 33a into which the parison 36 (refer to FIG. 6A) of a cylindrical shape which is made of a thermoplastic resin or the like is to be fitted and disposed are formed through a central portion thereof in the axial direction. The cavity 35 side (inside) of the fitting holes 32a and 33a is formed such that the diameter thereof is successively increased in a laterally directed tapering state such that they coincide with the inner diameter of the central die 31.

The core die 34 of the present embodiment exhibits a cylindrical shape. Accordingly, the internal shape of the core hole 31a is formed so as to correspond to the shape of the core die 34 of the cylindrical shape, and by the shape of the core die 34, the opening 23a of the communication section 23 is formed in a circular shape.

As shown in FIG. 6A, at the preparation step, the left side die 32, right side die 33 and core die 34 are combined with the central die 31, and then the parison 36 in a plasticized state is inserted and supported such that it extends through the fitting hole 32a of the left side die 32 and the fitting hole 33a of the right side die 33 thereby to place the parison 36 into the cavity 35.

As shown in FIG. 6B, at the molding step, one side of the parison 36 in the axial direction is closed up and pressurized fluid such as compressed air is injected into the inside of the parison 36 from the other side to inflate the parison 36 in the cavity 35, whereby the balloon 6 and the communication section 23 which is open to the outer face of the balloon 6 are integrally molded. Thereupon, the extrusion length of the core die 34 into the cavity 35 becomes the length of the communication section 23, and the outer diameter of the core die 34 becomes the inner diameter of the communication section 23. Thereafter, the molding die 30 is successively disassembled to take out the balloon 6 and the communication section 23 molded integrally with the balloon 6 as seen in FIG. 6C.

Then, at the attachment step, the balloon 6 and the communication section 23 molded integrally and the catheter main body 2 are connected to each other by a bonding agent or heat seal. At this time, the second lumen 4 of the inner tube 22 is communicated with the communication section 23 and the first lumen 3 is communicated with the internal space of the balloon 6. The second lumen 4 and communication section 23 and the first lumen 3 and internal space of the balloon 6 are isolated from each other without having a communicating portion therebetween.

### (Medical Treatment Method Using the Catheter 1)

Now, a medical treatment method of a vascular aneurysm using the catheter 1 is described taking a case where a vascular aneurysm which appears at a cerebral artery is treated.

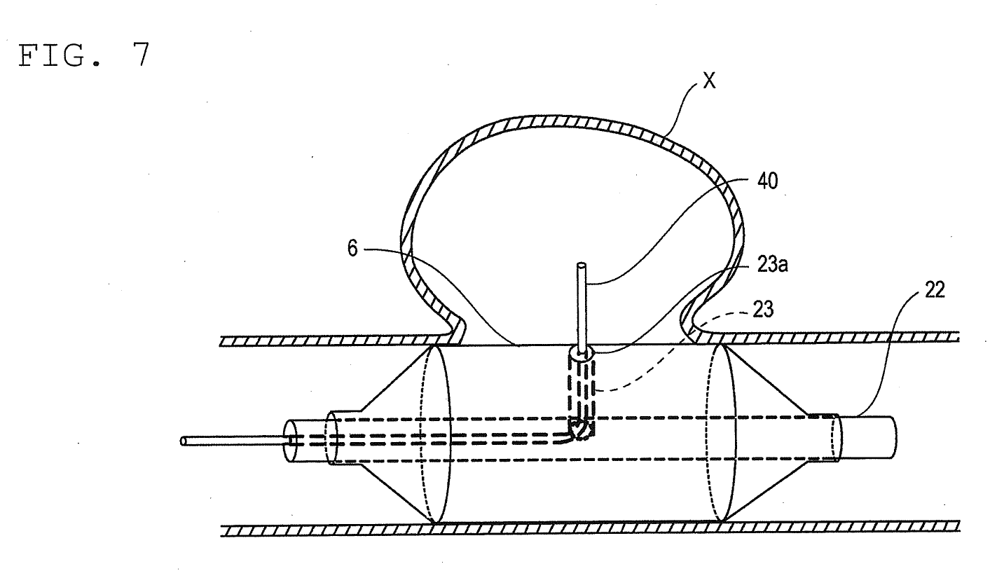

Generally describing with reference to FIG. 7, the medical treatment method of the present embodiment includes a catheter insertion step of inserting the catheter 1 into a blood vessel until the balloon 6 reaches a vascular aneurysm X, a blocking step of expanding, after the catheter insertion step, the balloon 6 to block the entrance of the vascular aneurysm X, and a filling step of inserting, after the blocking step, an instrument for the medical treatment from the communication section 23 into the vascular aneurysm X and filling the inside of the vascular aneurysm X using the instrument for the medical treatment.

At the catheter insertion step, the operator would indwell a sheath in the blood vessel, for example, by the Seldinger method and insert the catheter main body 2, in which a guide wire is fitted such that it projects by approximately 5 to 50 mm, into the blood vessel through the sheath and the guide catheter disposed in the sheath.

The operator would advance, while confirming the position of the distal end portion of the catheter 1 and the guide wire through the marker (not shown) provided at the distal end portion of the catheter 1 under X-ray radioscopy, the catheter 1 such that the guide wire precedes to the catheter 1 until the balloon 6 reaches the entrance of the vascular aneurysm X. After the balloon 6 reaches the entrance of the vascular aneurysm X, the operator would adjust the posture of the catheter 1 such that the opening 23a faces the inside of the vascular aneurysm X.

Then at the blocking step, the operator would feed liquid into the inside of the balloon 6 from the first opening 15 of the outer tube hub 11 through the first lumen 3 using a syringe or the like to expand the balloon 6. The expanded balloon 6 blocks up the entrance of the vascular aneurysm X with the cylindrical portion 6a thereof. In this state, although it is possible for the operator to remove the guide wire from the second opening 16 of the inner tube hub 12, usually the guide wire for a balloon catheter is left indwelling in the second lumen 4 of the balloon catheter. The indwelling guide wire can be used to advance a micro catheter hereinafter described along the lumen of the micro catheter when the micro catheter is transmitted. In this instance, if the guide wire is retracted a little such that the distal end thereof is inserted in the communication section 23, then it can be used for transmission of the micro catheter to the communication section 23. Also when the balloon catheter is drawn off, this guide wire can be used.

Thereafter, at the filling step, the operator would insert the micro catheter 40, which is a tube member open at the opposite ends thereof, from the second opening 16 using the guide wire to introduce the distal end of the micro catheter 40 into the vascular aneurysm X through the second lumen 4 and the communication section 23. At this time, the guide wire for the balloon catheter described above may be used to advance the micro catheter into the communication section or a guide wire for a micro catheter different from the guide wire for the balloon catheter may be used separately. After the introduction, the operator would remove the guide wire and supply a coil into the vascular aneurysm X through the micro catheter 40 to fill the vascular aneurysm X.

After the vascular aneurysm X is filled, the operator would draw off the micro catheter 40 from the second opening 16. Thereafter, the operator would suck and discharge the liquid from the first opening 15 and place the balloon 6 into the contacted or folded state and then draw off the catheter main body 2 from the blood vessel, thereby ending the procedure.

Working effects of the present embodiment are described.

With the catheter 1, since it is possible to insert, while the balloon 6 is kept in close contact with the aneurysm entrance, the micro catheter 40 into the vascular aneurysm X through the second lumen 4 and the communication section 23 to supply a coil, protrusion of the coil from the vascular aneurysm X can be prevented. Further, since not the micro catheter 40 is inserted between the aneurysm entrance and the balloon 6 and sandwiched but the micro catheter 40 can be inserted into the vascular aneurysm X through the second lumen 4 and the communication section 23, coming off of the micro catheter 40 arising from insufficient sandwiching as in the prior art does not occur. Further, with the catheter 1, there is no necessity to place both of a balloon catheter and a micro catheter independently of each other in a blood vessel, medical treatment of the vascular aneurysm can be carried out even if the blood vessel has a small inner diameter. Accordingly, the catheter 1 can carry out medical treatment of the vascular aneurysm X reliably and safely.

Further, the opening 23a of the communication section 23 is a small hole which restricts the change of the direction of the micro catheter 40 fitted in the opening 23a, and as a result, since the direction of the micro catheter 40 is less likely to change, a coil can be supplied stably into the vascular aneurysm X.

Further, according to the production method of the catheter 1 of the present embodiment, the catheter 1 with which a medical treatment of a vascular aneurysm can be carried out reliably and safely can be produced.

### Second Embodiment

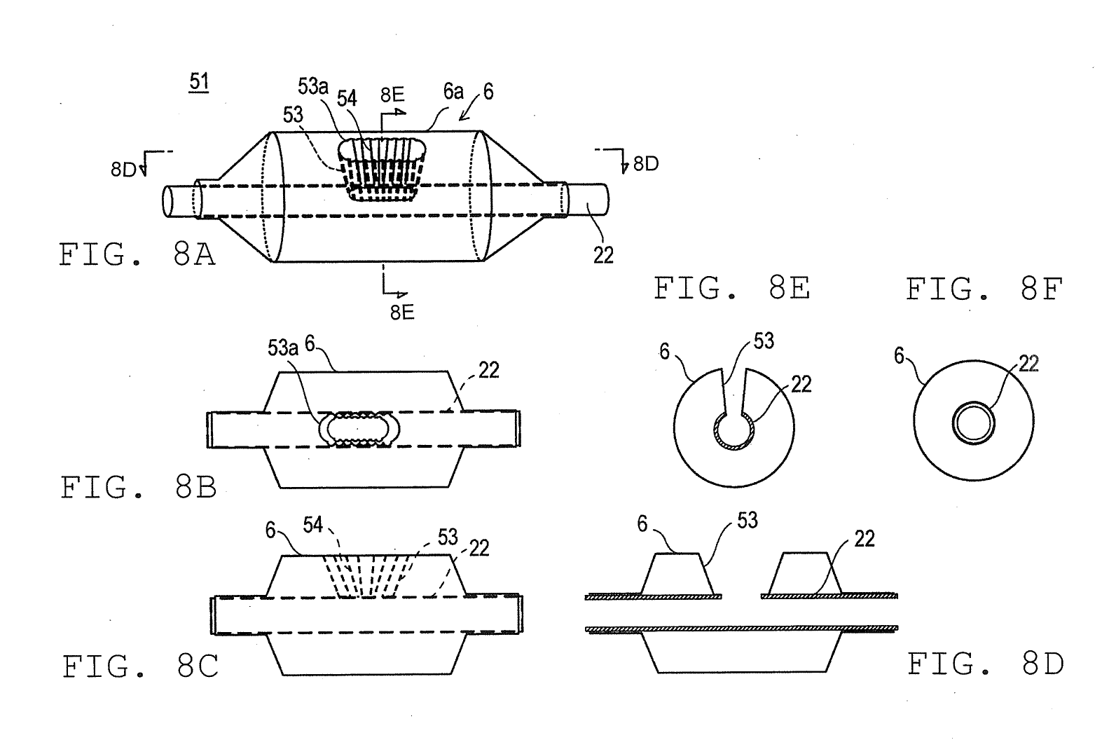

Describing generally with reference to FIGS. 8A-8F, although a second embodiment is substantially similar to the first embodiment, it is different from the first embodiment in the communication section 53. Since the other points of the second embodiment are similar to those of the first embodiment, an overlapping description of them is omitted. Further, components having same functions as those of the first embodiment are denoted by like reference characters.

As shown in FIGS. 8A-8D, the opening shape of an opening 53a of the communication section 53 in the second embodiment is formed as a shape of a hole elongated along an axial direction of the catheter main body 2. Further, on the inner face of the communication section 53, a groove 54 is formed which extends in a direction intersecting with an axial direction of the catheter main body 2. In the present embodiment, a plurality of grooves 54 are formed such that they project from an inner peripheral face, which faces the balloon lumen of the communication section 53 in the form of an elongated hole, toward the lumen of the communication section 53, and are arrayed in a direction substantially perpendicular to the axis of the catheter main body having an arcuate cross section. The grooves may be formed such that the width between adjacent ones of the grooves gradually increases from the inner tube 22 toward the balloon outer peripheral face. Therefore, the opening shape of the side face in the longitudinal direction of the opening 53a has a substantially wave shape formed from a plurality of arcs connected to each other.

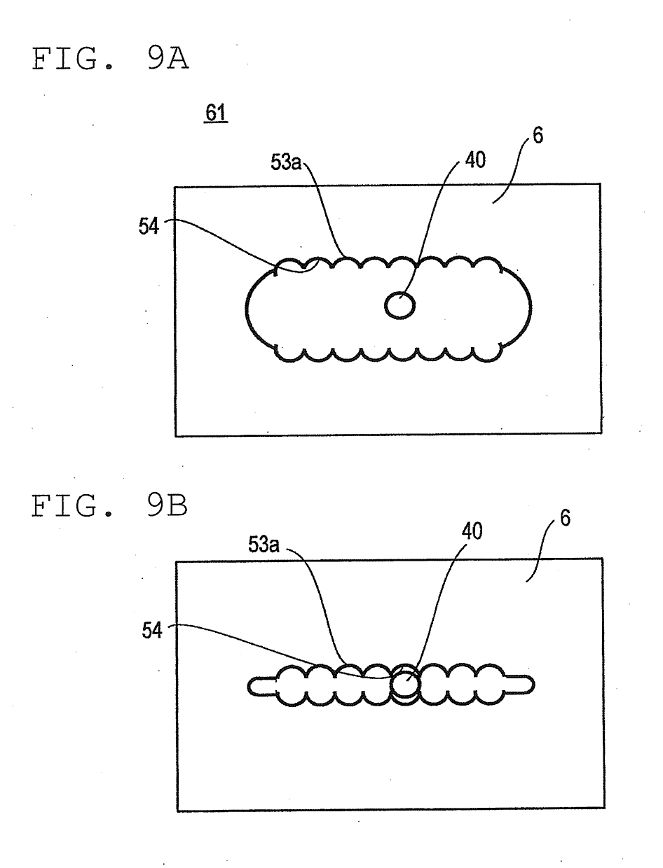

Further, as shown in FIGS. 9A and 9B, the balloon 6 of the second embodiment is different from that of the first embodiment in that liquid is supplied further into the balloon 6 in an expanded state to further expand the balloon 6 to close the opening 53a. By suitably selecting a material for molding the balloon 6, such a stepwise expansion can be achieved. The material from which the balloon 6 is configured includes, for example, nylon.

In the second embodiment, since the shape of the communication section 53 is different from that in the first embodiment, it is necessary to form the core die 34 of the molding die 30 such that the shape of the same corresponds to the shape of the communication section 53. Further, if the core die 34 is formed so that the shape thereof corresponds to the shape of the communication section 53 and the core hole 31a of the central die 31 is formed in a shape corresponding to that of the core die 34, then the production method of a catheter 51 can be carried out by steps similar to those in the first embodiment. Further, although, in the molding die 30 of the second embodiment, the central die 31 is formed in a cylindrical shape and the left side die 32 and the right side die 33 are formed in a shape of a disk, the shapes of them are not limited to them, but the central die 31, left side die 32 and right side die 33 may be formed in a shape split into two upper and lower or left and right molds such that the two-split type molds are combined from above and below or from the left and the right. Further, also the medical treatment method using the catheter 51 of the second embodiment is substantially similar to that of the first embodiment.

As described above, in the second embodiment, since the opening shape of the opening 53a of the communication section 53 is a shape of a hole elongated along the axial direction of the catheter main body 2, it is easy to supply a coil to an aimed place in the vascular aneurysm X by directing the micro catheter 40 inserted in the vascular aneurysm X from the communication section 53 toward a desired direction. Therefore, the second embodiment is superior in workability.

Further, since the opening 53a is closed to retain the micro catheter 40 in a desired direction, a coil can be supplied stably to an aimed place in the vascular aneurysm X.

Further, when the opening 53a is closed, since the micro catheter 40 is fitted in the groove 54, the micro catheter 40 is not likely to be crushed but is retained stably.

Further, since the catheter 51 of the second embodiment has a configuration similar to that of the catheter 1 of the first embodiment, it exhibits effects similar to those of the first embodiment.

### Third Embodiment

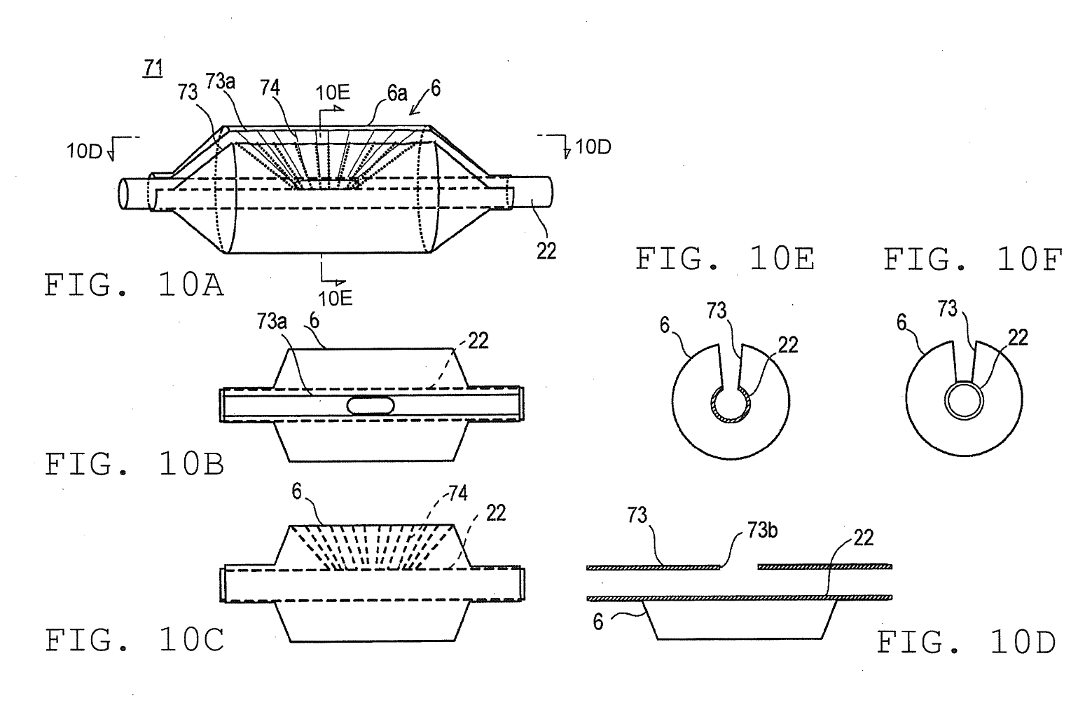

Generally describing with reference to FIGS. 10A-10F, although a third embodiment is substantially similar to the first embodiment, it is different from the first embodiment in a communication section 73. Since the other points of the third embodiment are similar to those of the first embodiment, an overlapping description of them is omitted. Further, components having similar functions to those of the first embodiment are denoted by like reference characters.

As shown in FIGS. 10A-10C, the opening shape of an opening 73a of the communication section 73 in the third embodiment is formed to be an elongated hole (elongated cavity) elongated along an axial direction of the catheter main body 2 and besides the opening 73a extends to an end portion of the balloon 6. In particular, an upper half of the balloon 6 in the third embodiment is formed in a leftwardly and rightwardly split form in a diametrical direction by the communication section 73, and the balloon 6 is formed such that the sectional shape thereof in a diametrical direction is a C shape.

Further, on the inner face of the communication section 73, similarly as in the second embodiment, a groove 74 which extends in a direction intersecting with an axial direction of the catheter main body 2, more particularly, a plurality of grooves 74 are formed such that they project from an inner peripheral face, which faces the balloon lumen of the communication section 73 in the form of an elongated hole, toward the lumen of the communication section 73, and are arrayed in a direction substantially perpendicular to the axis of the catheter main body having an arcuate cross section. The grooves may be formed such that the width between adjacent ones of the grooves gradually increases from the inner tube 22 toward the balloon outer peripheral face. Therefore, the opening shape of the side face in the longitudinal direction of the opening 73a of the communication section 73 has a substantially wave shape formed from a plurality of arcs connected to each other similarly as in the second embodiment.

Also in the third embodiment, liquid is supplied further into the balloon 6 in an expanded state to further expand the balloon 6 to close the opening 73a similarly as in the second embodiment.

In the third embodiment, since the shape of the communication section 73 is different from that in the first embodiment, it is necessary to form the core die 34 of the molding die 30 such that the shape of the same corresponds to the shape of the communication section 73. Further, if the core die 34 is formed so that the shape thereof corresponds to the shape of the communication section 73 and the shape of the central die 31, right side die 32 and left side die 33 is improved, then the production method of the catheter 71 can be carried out by steps similar to those in the first embodiment. Further, also the medical treatment method using the catheter 71 of the third embodiment is substantially similar to that of the first embodiment.

As described above, in the third embodiment, the opening shape of the opening 73a is a shape of an elongated hole elongated along the axial direction of the catheter main body 2, and besides extends to an end portion of the balloon 6. Therefore, it is easy to move the micro catheter 40 over a greater range to supply a coil to an aimed place in the vascular aneurysm X, and therefore, the operability is good.

Further, since the opening 73a is closed to retain the micro catheter 40 in a desired direction, a coil can be supplied stably to an aimed place in the vascular aneurysm X.

Further, when the opening 73a is closed, since the micro catheter 40 is fitted in the groove 74, the micro catheter 40 is not likely to be crushed but is retained stably.

Further, since the catheter 71 of the third embodiment has a configuration similar to that of the catheter 1 of the first embodiment, it exhibits effects similar to those of the first embodiment.

It is to be noted that the present invention is not limited to the embodiments described above but can be modified in various manners within the scope of the claims. For example, the present invention is not limited to applications to an aneurysm of a cerebral artery but can be applied widely to medical treatment applications of a vascular aneurysm.

Further, while, in the embodiment described above, a coil is filled into a vascular aneurysm through the micro catheter 40, the present invention is not limited to this, but agent may be filled into a vascular aneurysm through a micro catheter.

What is claimed is:

1. A catheter comprising:
a catheter main body including a first lumen configured for supplying and discharging expanding fluid therethrough and a second lumen configured for receiving an elongated instrument for medical treatment therein;
a balloon mounted at a distal end portion of said catheter main body and having an inflatable interior space that is in communication with said first lumen such that said balloon is expanded and contracted by supply and discharge of the expanding fluid into and out from the balloon interior space, respectively; and
a communication section having an opening generally at an outer face of said balloon and being configured to be in communication with said second lumen and to be isolated from the interior space of said balloon.

2. The catheter according to claim 1, wherein the opening of said communication section formed on the outer face of said balloon is a small hole which restricts a change of the direction of the instrument for the medical treatment fitted in said opening.

3. The catheter according to claim 1, wherein the catheter main body is elongated to extend in an axial direction, and the opening of said communication section formed on the outer face of said balloon is an elongated hole elongated along the axial direction of said catheter main body.

4. The catheter according to claim 3, wherein the opening of said communication section extends to an end portion of said balloon.

5. The catheter according to claim 3, wherein the balloon is of a predetermined material such that when additional expanding fluid is supplied into said balloon interior space with the balloon already in an expanded state, said balloon further expands to block the opening of said communication section.

6. The catheter according to claim 5, wherein said communication section has a groove formed on an inner face thereof that extends in a direction transverse to the axial direction of said catheter main body.

7. A method for medically treating a vascular aneurysm, the method comprising:
inserting a balloon catheter into a blood vessel until the balloon catheter is adjacent an opening of the vascular aneurysm;
expanding a balloon of the balloon catheter to block the opening of the vascular aneurysm;
inserting an instrument for medical treatment of the vascular aneurysm into a passageway of the balloon catheter that opens along an outer surface portion of the balloon adjacent the opening of the vascular aneurysm, and
filling the vascular aneurysm using the instrument for the medical treatment.

8. A production method of a catheter, the production method comprising:
placing a tube which has a hollow shape and is expandable, into a molding die which has a cavity and an extruding portion which projects into the cavity;
of injecting pressurized fluid into the tube to expand the tube to mold a balloon and a communication section open to an outer face of the balloon and isolated from an inflatable interior of the balloon; and
attaching the balloon and the communication section to a distal end portion of a catheter main body which includes a first lumen for supplying and discharging expanding fluid therethrough and a second lumen for receiving an elongated instrument for medical treatment to establish communication of the inflatable interior of the balloon with the first lumen and communication of the communication section with the second lumen.

9. A balloon catheter comprising:
an elongate catheter main body for being inserted into a blood vessel;
an expandable balloon having a flexible body and an expandable interior space of the body, the expandable balloon being carried on the catheter main body for being disposed adjacent a vascular aneurysm;
a first passageway of the catheter main body configured for supplying the expandable interior space of the balloon flexible body with fluid for expansion thereof for substantially blocking an opening of the vascular aneurysm; and
a second passageway of the catheter main body isolated from the balloon interior space and having an opening through the balloon flexible body to be aligned with the vascular aneurysm opening for receiving a medical treatment delivery instrument therethrough while the vascular aneurysm opening is blocked by the expandable balloon.

10. The balloon catheter of claim 9 wherein the first passageway comprises a first lumen extending axially in the elongate catheter main body and opening to the balloon interior space, and the second passageway comprises a second lumen extending axially in the elongate catheter main body to the balloon and a communication section to which the second lumen is open and which extends transverse to the second lumen and opens through the balloon flexible body.

11. The balloon catheter of claim 9 wherein the first and second passageways extend axially in the catheter main body with the second passageway extending axially beyond the first passageway.

12. The bottom catheter of claim 9 wherein the second passageway has a communication section including the opening through the balloon flexible body and which has portions of the balloon flexible body on either side thereof for being expanded to block the vascular aneurysm opening while a medical treatment is delivered into the vascular aneurysm by the medical treatment delivery instrument.

13. The balloon catheter of claim 9 wherein the second passageway opening through the balloon flexible body has a substantially fixed, small cross-sectional configuration to minimize shifting of the medical treatment delivery instrument when extending therethrough.

14. The balloon catheter of claim 9 wherein the balloon flexible body is of a predetermined material configured so that expansion of the expandable interior space causes the second passageway opening to substantially close to allow the balloon flexible body to be expanded against and to stably hold the medical treatment delivery instrument extending through the second passageway opening.

15. The balloon catheter of claim 14 wherein the second passageway opening is elongated.

16. The balloon catheter of claim 14 wherein the balloon flexible body extends along the second passageway opening and has grooves therein.

17. The balloon catheter of claim 14 wherein the balloon flexible body extends axially and has axially opposite ends, and the second passageway is axially elongated to extend to at least one of the balloon flexible body ends.

18. The method of claim 7 wherein the balloon is expanded by supplying the balloon with fluid through another passageway in the balloon catheter that opens to an expandable interior space of the balloon and which is isolated from the balloon catheter passageway through which the instrument is inserted.

19. The method of claim 7 wherein expanding of the balloon includes expanding balloon portions on either side of the balloon catheter passageway through which the instrument is inserted.

20. The method of claim 7 wherein expanding the balloon includes engaging the instrument extending through the balloon catheter passageway with the expanded balloon for holding the instrument steady during the filling of the vascular aneurysm.
